(19) United States
(12) Patent Application Publication
(10) Pub. No.: US 2025/0195113 A1
(43) Pub. Date: Jun. 19, 2025

(54) SPINAL IMPLANT DELIVERY DEVICE FOR PROTECTING TISSUE

(71) Applicant: Amplify Surgical, Inc., Irvine, CA (US)

(72) Inventors: Dong-Hwa Heo, Seoul (KR); Andy Wonyong Choi, Irvine, CA (US)

(21) Appl. No.: 18/987,830

(22) Filed: Dec. 19, 2024

Related U.S. Application Data
(60) Provisional application No. 63/611,913, filed on Dec. 19, 2023.

(51) Int. Cl. A61B 17/70; A61B 17/00; A61B 17/56; A61B 90/00

(52) U.S. Cl. CPC A61B 17/7061; A61B 90/03; A61B 90/08; A61B 90/361; A61B 2017/00367; A61B 2017/00902; A61B 2017/564; A61B 2090/036; A61B 2090/0807

(57) ABSTRACT

A spinal implant delivery device can include a pair of insertion members that can be inserted into a subject towards, for example, an intervertebral space or other target site for a spinal implant. The insertion members can define a channel with an open side and a slot through which an instrument or implant cannot exit the channel. The slot thereby protects adjacent tissue and nerves from potential damage during a surgical procedure. The insertion members can include distance indicators and/or a plurality of apertures. The distance indicators can be used to visually determine how deep the insertion members are inserted into a subject at any given time. The apertures can provide a direct view into the channel of the spinal implant delivery device, allowing a surgeon or other user to visually determine the position of an instrument or implant disposed in the channel.

### CROSS-REFERENCE TO RELATED APPLICATION

The present application claims the benefit of U.S. Provisional Patent Application No. 63/611,913, filed Dec. 19, 2023, the disclosure of which is incorporated herein by reference in its entirety.

### TECHNICAL FIELD

The present disclosure relates generally to medical systems and, more particularly, to systems, devices, and methods for delivering implants while protecting tissue, such as nerve tissue.

### BACKGROUND

Individuals often suffer from damaged or displaced spinal discs and/or vertebral bodies due to trauma, disease, degenerative defects, or wear over an extended period of time. One result of this displacement or damage to a spinal disc or vertebral body may be chronic back pain. A common procedure for treating damage or disease of the spinal disc or vertebral body may involve partial or complete removal of an intervertebral disc. An intervertebral implant (commonly referred to as an interbody spacer or cage) can be inserted into the cavity created where the intervertebral disc was removed to help maintain height of the spine and/or restore stability to the spine. An interbody spacer may also provide a lordotic correction to the curvature of the spine. An example of an interbody spacer that has been commonly used is a fixed dimension cage, which typically is filled with bone and/or bone growth-inducing materials. Unfortunately, it may be difficult to implant the interbody spacer at the intended implantation site between vertebral bodies. Additionally, conventional surgical techniques can cause a significant amount of trauma at or near the implantation site (e.g., injury to nerve tissue), which can significantly increase recovery time and/or lead to patient discomfort. Accordingly, there is a need for improved surgical systems, visualization techniques, and/or related technologies for delivering a spinal implant.

### BRIEF DESCRIPTION OF THE DRAWINGS

FIG. 1A is a side view of a spinal surgical system in accordance with an embodiment of the disclosure.

FIG. 1B is a side view of a spinal surgical system in accordance with another embodiment of the disclosure.

FIG. 2A is a schematic top plan view showing surgical approaches to a lumbar spine for performing procedures.

FIG. 2B is an isometric view of the lumbar spine of FIG. 2A.

FIGS. 3A, 3B, 3C, and 3D are front isometric, rear isometric, top, and bottom views, respectively, of a spinal implant delivery device in accordance with an embodiment of the disclosure.

FIGS. 3E and 3F are enlarged bottom isometric and side views, respectively, of portions of the spinal implant delivery device of FIG. 3A.

FIG. 4 is a front view of a portion of the spinal implant delivery device of FIG. 3A in an uncompressed state and in a compressed state.

FIGS. 5A and 5B illustrate surgical steps for performing spinal procedures in accordance with embodiments of the disclosure.

FIGS. 6A and 6B are top views of a spinal surgical system including the spinal implant delivery device of FIG. 3A in different orientations in accordance with embodiments of the disclosure.

FIGS. 7A and 7B are side views of the spinal surgical system of FIGS. 6A and 6B, respectively.

FIGS. 8A and 8B are superior-to-inferior views of the spinal surgical system of FIGS. 6A and 6B, respectively.

FIG. 9 is another superior-to-inferior view of the spinal surgical system of FIG. 6A.

### DETAILED DESCRIPTION

The following disclosure describes various embodiments of delivery devices and associated systems and methods of use. The delivery device can include slotted or elongated split delivery guide that can be inserted into a subject towards, for example, an intervertebral space or other target site for delivering one or more spinal implants. The split elongated delivery guide can include spaced apart insertion members defining a channel along which implants can be passed. The insertion members can be compressed (e.g., pushed towards each other) for insertion into the patient. When uncompressed, sidewalls of the insertion members can extend in parallel directions and define a generally U-shaped delivery channel through which a spinal implant can be delivered. The insertion members can each have a geometry (e.g., an L-shaped cross-section) that defines a channel sidewall and channel bottom. The channel sidewalls can define an upper opening of the channel. The insertion members thereby protect adjacent tissue and nerves from potential damage while the implant is delivered along the channel. For example, the delivery device can be oriented such that the protected side or face of the delivery devices faces a particular area of the subject's tissue (e.g., nerve tissue), organs, vascular features, or the like. In another example, the device can be inserted into the subject with the protected side facing the spinal cord to deliver an intervertebral cage distally past the spinal cord and into the disc space. The split elongated delivery guide can prevent physical contact between the cage and the spinal cord.

In some embodiments, the insertion members include distance indicators and/or a plurality of apertures. The distance indicators can be used to visually determine how deep the insertion members are inserted into a subject at any given time. The apertures can provide a direct view into the channel of the spinal implant delivery device, allowing a surgeon or other user to visually determine the position of an instrument or implant disposed in the channel. In some embodiments, the apertures are aligned with the distance indicators such that the surgeon or other user can more accurately determine the position of the instrument or implant.

The spinal implant delivery device can be included in a spinal surgical system that also includes a visualization instrument, which can help a user identify tissue and operate the spinal implant delivery device while preventing or limiting injury or damage to non-targeted organs and tissues. In endoscopic-assisted surgeries, instruments and implantable devices can be precisely positioned using minimally invasive techniques to improve outcomes and reduce recovery times. Certain details are set forth in the following description and in the figures to provide a thorough understanding of such embodiments of the disclosure. Other details describing well-known structures and systems often associated with, for example, surgical procedures are not set forth in the following description to avoid unnecessarily obscuring the description of various embodiments of the disclosure.

### A. OVERVIEW

At least some embodiments are directed to spinal implant delivery devices and associated spinal surgery systems. The systems, devices, and methods disclosed herein can be used to implant a fixed or expandable interbody device (e.g., devices to space apart vertebral bodies, restore stability of the spine, provide lordotic correction, etc.), or perform other surgical procedures while protecting surrounding tissue and nerves.

In some embodiments, a spinal implant delivery device includes a handle assembly and an elongated split delivery guide coupled to the handle assembly. The elongated split delivery guide can include a first insertion member and a second insertion member extending in the same direction (i.e., parallel to the first insertion member). The first and second insertion members can be spaced apart to define a delivery channel. The first and second insertion members can include L-shaped cross-sections facing each other such that the channel has an opening at the top and a longitudinally-extending slot at the bottom. An instrument or implant disposed in the channel can exit the channel through the opening. The slot can have a sufficiently narrow gap so that an instrument or implant disposed in the channel cannot exit the channel through the bottom. The bottom can thereby prevent the instrument or implant from pressing against tissue or nerves on the other side of the slot, thereby protecting the tissue and nerves. The orientation of the device can be selected so that the slot faces tissue and nerves deemed to be particularly susceptible to damage depending on the type of surgical procedure, the location of the target site, the type of implant to be delivered, the angle at which the device is inserted into the subject, etc. In some embodiments, the handle assembly can assist in manual handling of the device and visually indicate the orientation of the device.

In some embodiments, the device includes distance indicators that can help a surgeon or other user visually determine how deep the device has been inserted into the subject. In some embodiments, the device includes apertures that allow the surgeon or other user to see through and visually determine the position of the implant or instrument disposed in the channel of the device. The apertures can be aligned with the distance indicators so that the position of the implant or instrument can be more accurately determined.

In some embodiments, a method for delivering a spinal implant to a subject's spine includes inserting a spinal implant delivery device in a subject, and delivering a spinal implant to the subject's spine through a chamber defined by the spinal implant delivery device. In some embodiments, the device can include an elongated split delivery device with a pair of insertion members extending longitudinally. The elongated split delivery device can be configured to bias the insertion members to be spaced apart by a particular distance. In some procedures, the elongated split delivery device can be manually squeezed to bring the two insertion members closer to one another prior to insertion of the device into the subject, then released when the device is properly inserted to create an enlarged space for implant delivery.

In some embodiments, the spinal implant delivery device is used in a spinal surgical system that also include a visualization instrument and/or other devices to assist the surgical procedure, including instrument holders, pillows, etc. Surgical techniques described herein can include a spinal implant delivery procedure, a decompression procedure, an oblique lumbar interbody fusion (OLIF) procedure, a lateral lumbar interbody fusion (LLIF) procedure, a posterior lumbar interbody fusion (PLIF) procedure, a transforaminal lumbar interbody fusion (TLIF) procedure, an anterior lumbar interbody fusion (ALIF) procedure, or combinations thereof.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

### B. SPINAL SURGICAL SYSTEMS

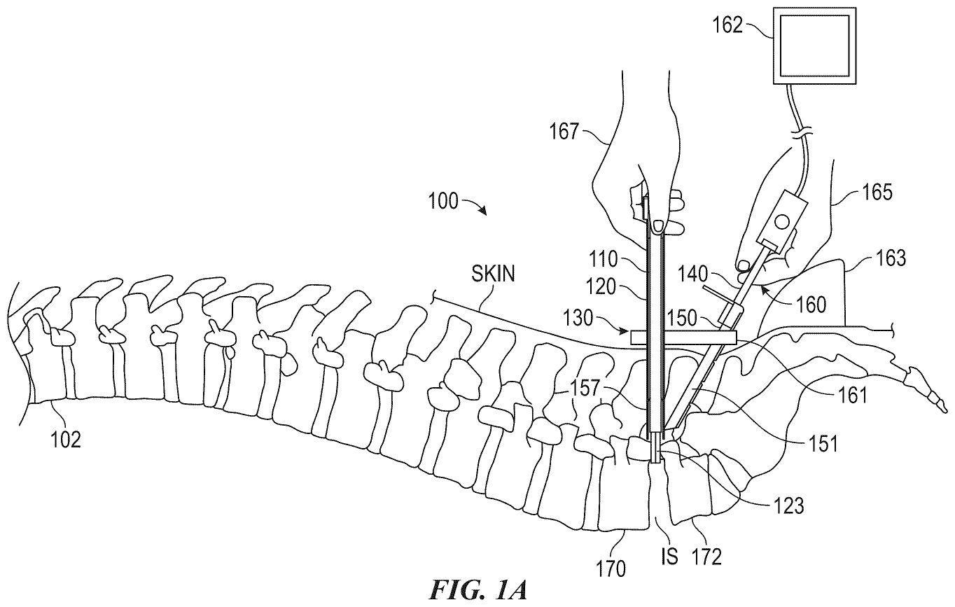

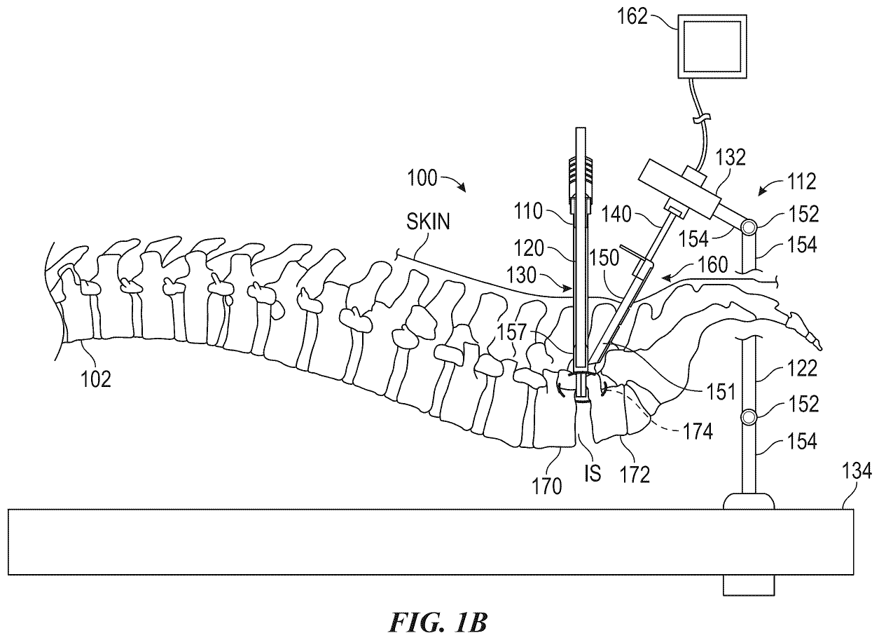

FIG. 1A a side view of a spinal surgical system 100 (“system 100”) positioned along a human subject's spine 102 in accordance with an embodiment of the disclosure. The system 100 can include an instrument assembly 130, a visualization assembly 160, and a multi-portal instrument holder 161 (“instrument holder 161”). The instrument holder 161 can hold the instrument assembly 130 and visualization assembly 160. The instrument holder 161 can be configured to hold components (e.g., cannulas, surgical instruments, combinations thereof, etc.) of the instrument assembly 130, visualization assembly 160, and/or other equipment, such as surgical beds.

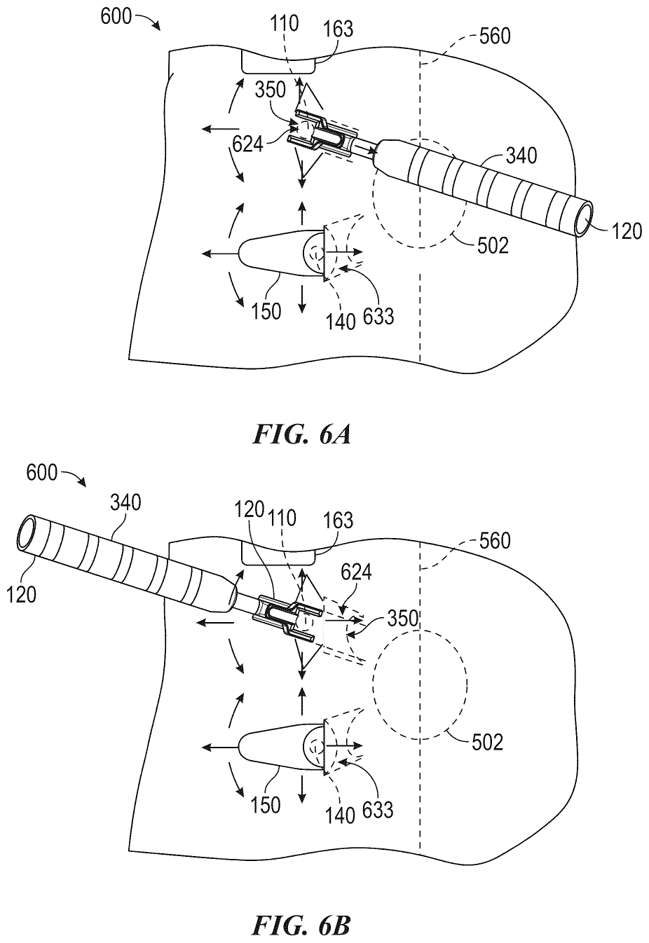

The instrument assembly 130 can an implant delivery device 120 (“the device 120”) defining a delivery path 121 along which an implant 123 carried by an instrument 110 can be delivered toward the implantation site IS. The device 120 can be slotted or be a split elongated delivery guide that can be inserted into a subject towards, for example, an intervertebral space or other target site for delivering the implant 123. The visualization assembly 160 can include a visualization instrument 140 and a cannula or device 150. In some embodiments, instruments 110, 140 include instruments disclosed in U.S. application Ser. No. 17/902,685 and U.S. Pat. No. 11,678,906. The instruments 110 and 140 can be moved distally and/or laterally out of the devices 120 and 150, respectively, which can be positioned in incisions or endoscopic ports, to access a relatively large working space along the patient's spine 102. In some embodiments, the devices 120 and 150 are identical or generally similar. In other embodiments, the devices 120 and 150 are different. The devices 120, 150 can have longitudinally extending openings along their entire lengths or portion thereof (not fully visible in FIG. 1A) to allow distal portions of the respective instruments 110, 140 to be moved laterally into and out of sides of the devices 120, 150. Positioning of instruments is discussed in connection with FIGS. 6A-8B.

The illustrated device 120 has an open top facing away from the subject's spine (the open side is partially visible and partially obscured by the instrument 110 in FIG. 1A) and a protected side or face opposite the open side and facing the subject's spine (the protected side is not visible in FIG. 1A). The protected side can include a backside atraumatic surface 157 such that contact with the device 120 and/or the instrument 110 limits, reduces, or substantially eliminates trauma to tissue, nerves, and/or other components around the spine 102. A series of instruments can be delivered through the device 120. In some procedures, the instrument 110 can be used to remove tissue (e.g., intervertebral disc, tissue contributing to stenosis, etc.), form access paths to implantation sites, prepare an implantation site by, for example, moving organs or tissue (e.g., moving nerve tissue), prepare vertebral bodies (e.g., roughening or shaping vertebral endplates), or the like. In some embodiments, the device 120 can create an enlarged intervertebral space at the target site, as will be described in further detail herein. An interbody implant can be delivered through the device 120 (e.g., via the instrument 110) to the intervertebral space IS. In some embodiments, a different cannula or device can be used for guiding the instrument 110 while the device 120 is used to deliver the implant 123. In expandable implant embodiments, an expandable interbody fusion implant 123 can be expanded to push apart vertebral endplates.

With continued reference to FIG. 1A, a customizable ergonomic pillow 163 can be used to position a user's body during the surgical procedure. The illustrated pillow 163 is configured to support the user's hand 165 holding the visualization assembly 160. Any number of pillows can be used during the surgical procedure to support the user's other hand 167 or other body parts. Additionally, sets of pillows can be used for corresponding sets for surgical actions. For example, a first set of pillows can be used to perform a first decompression procedure at a first level of the patient's spine, a second set of pillows can be used to perform a second decompression procedure at a second level of the patient's spine, and a third set of pillows can be used to perform one or more implantation procedures at the first and/or second level or other levels. The pillows can be coupled to the patient's body, operating table, and/or other attachment features.

The visualization assembly 160 can provide intraoperative endoscopic viewing of workspaces, delivery paths, organs, tissue (e.g., nerve tissue) implantation sites, implants, interbody fusion devices (e.g., before, during, and/or after delivery), instrument(s) (including dispensers, dilators, decompression instruments, etc.), and other areas or features of interest. The position of the devices 120, 150 can be selected based on the procedure and optical characteristics (e.g., field of view, zoom capability, etc.) of the visualization assembly 160. The visualization assembly 160 can be moved throughout the procedure to provide intraoperative endoscopic viewing of one, multiple, or all of the surgical steps. For example, the visualization assembly 160 can be used to view delivery of the spinal implant along the device 120, tissue contributing to nerve compression caused by narrowing of the spinal canal associated with arthritis of the spine, degeneration of spinal discs, and thickening of ligaments. Arthritis of the spine often leads to the formation of bone spurs, which can narrow the spinal canal and press on the spinal cord. This tissue can be viewed using the visualization assembly 160. In spinal disc degeneration, the visualization assembly 160 can view the inner tissue of the disc protruding through a weakened fibrous outer covering of the disc and pressing on the spinal cord and/or spinal nerve roots. The protruding tissue can be viewed before and/or during removal. The visualization assembly 160 can be used to also view ligaments pressing on the spinal cord and/or nerve roots to assist in treatment.

The visualization instrument 140 can be a low-profile fiber-optic endoscope positioned directly through an incision, an endoscopic port, or the like. The visualization instrument 140 can include one or more endoscopes having, without limitation, fiber optics (e.g., optical fibers), lenses, imaging devices, working lumens, light source controls, or the like for direct viewing or viewing via a display 162 (e.g., an electronic screen, a monitor, etc.). In some embodiments, the visualization instrument 140 can include a lumen through which fluid flows to irrigate the surgical site. For example, saline, or another suitable liquid, can be pumped through the visualization instrument 140 to remove tissue (e.g., loose tissue, bone dust, etc.) or other material impairing visualization. The visualization instrument 140 can also include one or more lumens (e.g., irrigation return lumens, vacuum lumens, etc.) through which the irrigation liquid can be withdrawn.

The visualization instrument 140 can illuminate the body cavity and enable high-resolution video visualization. A light source (e.g., a laser, light-emitting diode, etc.) located near or at the proximal end of the fiber optics can be used to transmit light to the distal end and provide illuminating light. This enables a surgeon to safely navigate into the subject's body and to illuminate specific body anatomy to view vertebral spacing, vertebral structures, nerves, bony buildup (e.g., buildup that could be irritating and pressing against nerves contributing to nerve compression), etc. This also allows a surgeon to illuminate portions of the device 120 and view a position of an implant along the length of the device 120. In some embodiments, visualization optics for vision and illumination are included within the distal tip of the visualization instrument 140. The configuration and functionality of the visualization instrument 140 can be selected based on the desired field of view, viewing resolution, pan/zoom functionality, or the like. Irrigation techniques, visualization devices, instruments, cannulas, and visualization and surgical techniques are discussed in U.S. application Ser. No. 17/902,685 and U.S. application Ser. No. 16/687,520, which are incorporated by reference in their entireties.

To position the devices 120, 150, the devices 120, 150 can be inserted into entrances formed in the subject's skin. The multi-portal instrument holder 161 can be adjusted to hold the devices 120, 150 at the fixed or altered positions while instruments (e.g., instruments 110, 140) are delivered through the devices 120, 150. The instrument holder 161 can be used to set the distance between the devices 120, 150 and can be locked to hold the devices at, for example, a set distance and/or angular orientation. For example, the instrument holder 161 can have locking mechanisms that are locked by the user to hold the devices 120, 150 stationary relative to one another. The instrument holder 161 can be unlocked to reposition the devices. This process can be performed any number of times to reposition the devices 120, 150.

The instrument holder 161 can also be used to hold the instruments 110, 140 in a similar manner. In some embodiments, an instrument holder or devices 120, 150 and another multi-portal instrument holder holds the instruments 110, 140. This allows for flexibility during the surgical procedure to hold various components stationary relative to one another when desired. For example, a multi-portal instrument holder in the form of a triangulation guide can be used with the instruments 110, 140. A multi-portal instrument holder can hold the devices 120, 150 stationary relative to one another. This allows for some instruments to be moved relative to one another while other instruments are held relatively stationary.

FIG. 1B is a side view of the spinal surgical system 100 in accordance with another embodiment of the disclosure. The system 100 of FIG. 1B is generally similar to the system 100 of FIG. 1A except as detailed below. The system 100 can include an instrument holder 112 having an arm 122 configured to be coupled to a structure 134 (e.g., surgical bed, a mounting bar, etc.) via, for example, one or more clamps, and a clamp or jaw 132 (“jaw 132”) coupled to the arm 122. The jaw 132 is configured to grip the visualization instrument 140 when the arm 122 is at a fixed configuration, thereby holding the visualization instrument 140 stationary relative to a patient. The arm 122 can include one or more joints (e.g., manual joints, motorized joints, etc.), linkages, motors, controllers, sensors, or the like. The configuration of the arm 122 can be selected based on the desired range of motion, degrees of freedom, or the like. In some embodiments, the arm 122 includes multiple joints 152 and linkages 154. The number of linkages, joints, and other components of the arm 122 can be selected based on the desired positioning of the instrument and range of motion.

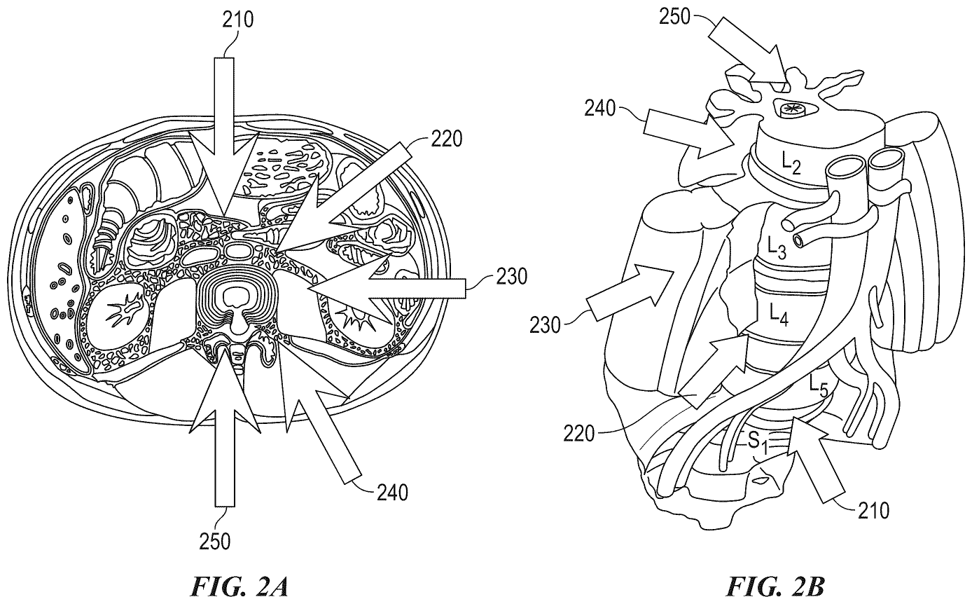

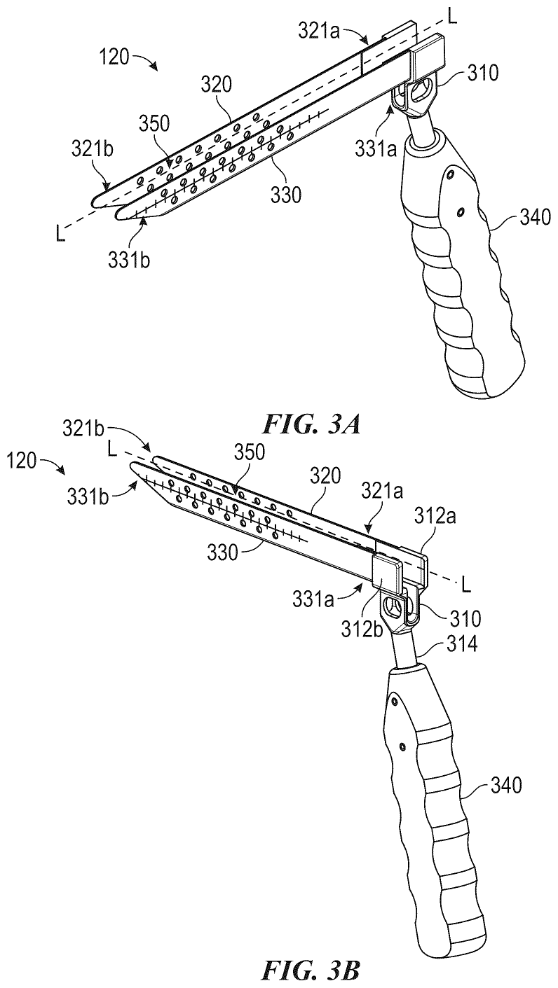

FIG. 2A is a schematic top plan view showing surgical approaches to a lumbar spine for performing procedures. FIG. 2B is an isometric view of the lumbar spine of FIG. 2A. The surgical approaches illustrated can be for performing procedures suitable for the system 100 of FIGS. 1A and 1B, and other systems disclosed herein. The number and configuration of physician-support pillows can be selected based on the spinal approach.

Referring to FIGS. 2A and 2B together, surgical equipment (e.g., the device 120) can be delivered via different paths, including an ALIF path 210, an OLIF path 220, an LLIF or extreme lateral lumbar interbody fusion (XLIF) path 230, a TLIF path 240, and a PLIF path 250. These paths can be used to deliver spinal implants to a target site on a subject's spine. These paths can also be used to perform other procedures disclosed herein. For example, one or more of the paths 210, 220, 230 240, 250 can be selected for multi-portal endoscopic approaches to perform a wider array of lumbar spine procedures than conventional one-portal techniques. Devices can be positioned along the same path or different paths to allow for independent positioning and manipulation of the endoscopic camera of the surgical instruments, thereby providing greater flexibility and enhanced visualization of spinal anatomy.

Surgical instruments can remove tissue to define working space(s) inside the patient. In one example TLIF procedure, the transforaminal path 240 may be employed to implant a single small expandable or non-expandable interbody spacer at the intervertebral space. In one example PLIF procedure, two interbody spacers can be delivered along the posterior path 250 and implanted at the intervertebral space. The two interbody spacers can cooperate to keep the vertebral bodies at the desired spacing and may be larger than the TLIF spacer. Additionally, multiple interbody spacers can provide lordotic correction by providing support at different heights. In one example LLIF procedure, a single, relatively large interbody spacer can be delivered along the lateral path 230 and implanted to provide asymmetrical support. In one example ALIF procedure, an asymmetric interbody spacer can be delivered along the anterior path 210 to provide support consistent with lordosis at that portion of the spine. Lateral approaches, transforaminal approaches, and anterior approaches can be used to access the cervical spine, thoracic spine, etc. The number of instruments, configurations of instruments, implants, and surgical techniques can be selected based on the condition to be treated.

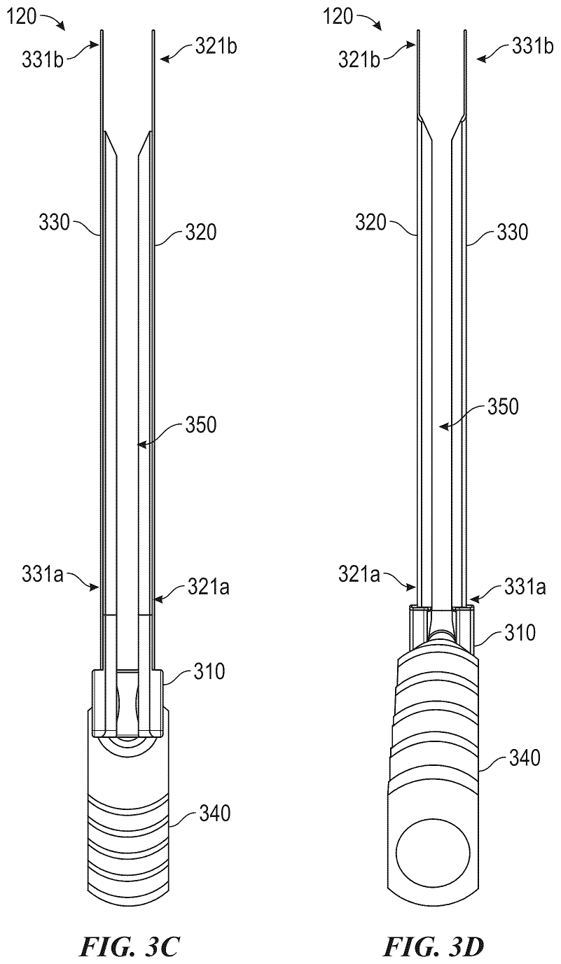

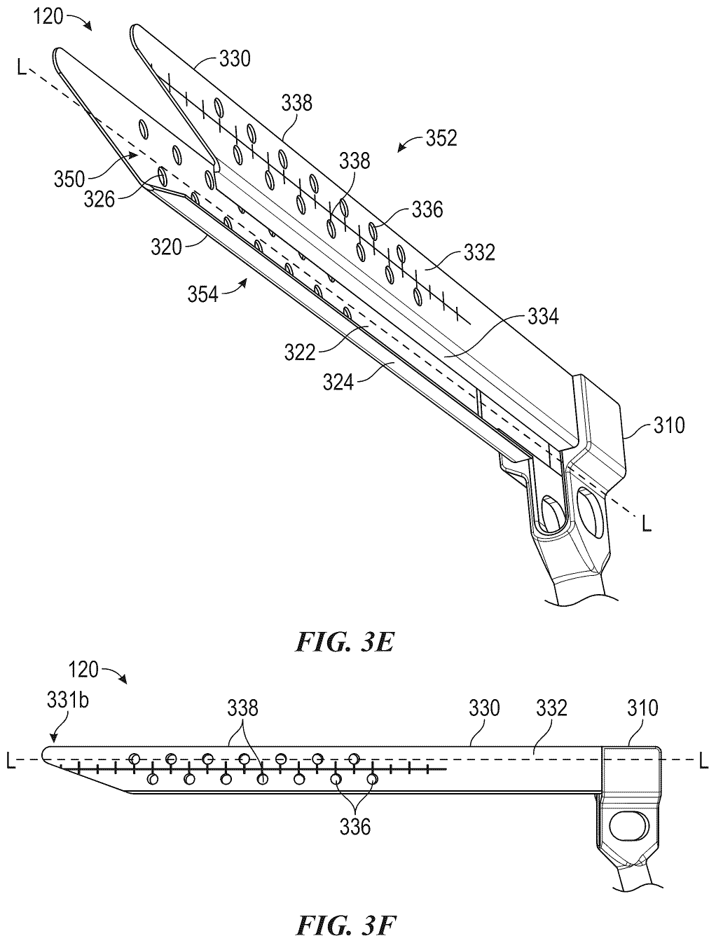

FIGS. 3A, 3B, 3C, and 3D are front isometric, rear isometric, top, and bottom views, respectively, of the implant delivery device 120 in accordance with an embodiment of the disclosure. Referring first to FIGS. 3A-3D together, the device 120 includes a handle assembly 340 and an elongated split delivery guide 310 (e.g., having a slotted channel member) coupled to the handle assembly 340 and defining a delivery channel 350 (e.g., an implant delivery channel). The elongated split delivery guide 310 can include a pair of cantilevered fingers, including a first insertion member 320 and a second insertion member 330 extending in a direction along a longitudinal axis L-L. The elongated split delivery guide 310 (including the first and second insertion members 320, 330) can be made from surgical- and/or medical-grade materials including metal (e.g., stainless steel, titanium), plastics, silicone, and/or other suitable materials. The handle assembly 340 can be made from metal, plastics, silicone, and/or other suitable materials.

As best shown in FIG. 3B, the elongated split delivery guide 310 can have a generally U-shaped geometry (e.g., cross-section) with a first pushing portion 312a and a second pushing portion 312b. The first and second insertion members 320, 330 can extend from the first and second pushing portions 312a, 312b, respectively. The handle assembly 340 can be coupled to a proximal and lower portion 314 of the elongated split delivery guide 310 and can extend at an angle relative to the first and second insertion members 320, 330. As will be described in further detail below, when the device 120 is inserted into a subject, the handle assembly 340 can provide a visual indicator of the orientation of the device 120.

The first insertion member 320 includes a proximal end portion 321a that is coupled to the first pushing portion 312a, and the second insertion member 330 includes a proximal end portion 331a that is coupled to the second pushing portion 312b. The first insertion member 320 also includes a distal end portion 321b opposite the proximal end portion 321a, and the second insertion member 330 includes a distal end portion 331b that is opposite the proximal end portion 331a. In the illustrated embodiment, the distal end portions 321b, 331b comprise free, cantilevered, and tapered tips configured to pierce and/or be inserted into a subject. Accordingly, the first and second insertion members 320, 330 can also be referred to herein as a pair of tapered distal sidewalls spaced apart from one another. The first and second insertion members 320, 330 also at least partially define a chamber extending along the longitudinal axis L-L and through which a spinal implant can be delivered, as will be described in further detail below. As an implant is delivered, the first and second insertion members 320, 330 can keep the implant within the channel 350.

FIGS. 3E and 3F are enlarged bottom isometric and side views, respectively, of portions of the device 120. Referring first the FIG. 3E, the first insertion member 320 includes a first portion 322 (e.g., a first sidewall) and a second portion 324 (e.g., a first bottom) extending substantially perpendicular to the first portion 322. Similarly, the second insertion member 330 includes a third portion 332 (e.g., a second sidewall) and a fourth portion 334 (e.g., a second bottom) extending substantially perpendicular to the third portion 332. The first portion 322 and the third portion 332 can be generally flat and parallel to one another. The second portion 324 and the fourth portion 334 can be generally flat and extend toward one another while defining a longitudinally-extending slot 354 that extends along the longitudinal axis L-L therebetween. In some embodiments, the first and second portions 322, 324 form a first L-shape, and the third and fourth portions 332, 334 form a second L-shape such that the first and second insertion members 320, 330 each comprise elongate structures with an L-shaped cross-section. Thus, a top of the channel 350 has an open side 352 through which an instrument and/or implant can exit, and a bottom of the channel 350 includes the slot 354 that prevents any instrument and/or implant above a predetermined size (based on the gap of the slot 354) from exiting. Accordingly, the elongated split delivery guide 310, as a whole, can have a generally U-shaped cross-section taken perpendicular to the longitudinal axis L-L.

Referring next to FIGS. 3E and 3F together, each of the first and second insertion members 320, 330, and more particularly, the first and third portions 322, 332 can include a first plurality of apertures 326 and a second plurality of apertures 336, respectively. The first and/or third portions 322, 332 can also include distance indicators 338 along the longitudinal axis L-L. In the illustrated embodiment, the distance indicators 338 include marks labeled with specific distance measurements (e.g., in 1 mm, 2 mm, 5 mm, 10 mm, or 15 mm increments from the distal end portion 331b). The apertures 336 can be aligned with and/or arranged along the distance indicators 338, as shown.

In operation, the tapered tips of the distal end portions 321b, 331b can pierce through the subject's skin and/or tissue as the device 120 is inserted into the subject. The distance indicators 338 can visually signal to a surgeon or other user of the device 120 how deep the device 120 has been inserted into the subject. For example, the distance indicator 338 at the subject's skin surface can indicate the total depth that the device 120 has been inserted. In another example, a visualization instrument (e.g., the visualization instrument 140) can be used to determine the position of the device 120 based on the distance indicators 338. As a spinal implant is delivered through the channel 350, the visualization instrument can provide viewing through the apertures 326, 336 to view the position of the implant relative to the device 120. The apertures 326, 336 can be particularly advantageous in embodiments in which the first and second insertion members 320, 330 are made from opaque materials (e.g., metal). However, the apertures 326, 336 can still be particularly in embodiments in which the first and second insertion members 320, 330 are made from transparent or translucent materials (e.g., plastics), such as by providing a clearer through-view of the implant being delivered. Therefore, the apertures 326, 336 and the distance indicators 338 can assist a surgeon or other operator during an implant procedure or other surgical procedure in positioning and/or orienting the device 120 and delivering the implant to the target site with precision.

As mentioned above and discussed in greater detail below, the bottom of the device 120 including the slot 354 can prevent from any instruments and/or implant disposed in the channel 350 from moving or angling out of the channel 350, thereby protecting adjacent tissue and nerves. In the illustrated embodiment, the handle assembly 340 is oriented on the side of the slot 354 and away from the open side 352 (see FIGS. 3A and 3B), allowing a surgeon or other user to conveniently determine which side of the device 120 includes the slot 354 when the device is, for example, inserted in a subject. In other embodiments, the handle assembly 340 can be oriented on the open side 352 and away from the side of the slot 354.

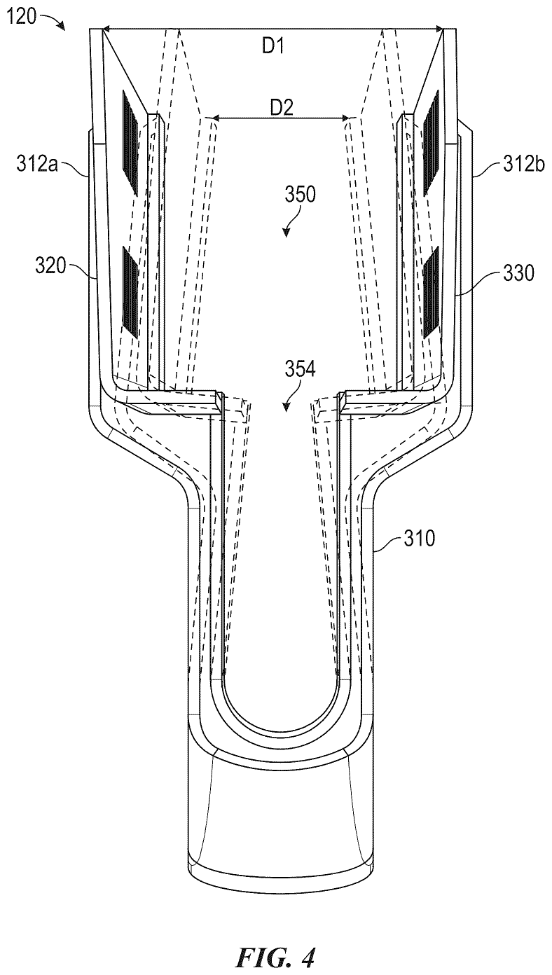

FIG. 4 is a front view of a portion of the device 120 in an uncompressed or neutral state and in a compressed state (in phantom lines). When the device is in the neutral state, the first and third portions 322, 332 of the first and second insertion members 320, 330, respectively, are oriented parallel to one another and are separated by a first distance D1. In some embodiments, the elongated split delivery guide 310 has a stiffness or elasticity that allows the elongated split delivery guide 310 to bias the first and second insertion members 320, 330 to remain separated by the first distance D1. Prior to inserting the device 120 in a subject, the first and second pushing portions 312a, 312b of the elongated split delivery guide 310 are movable toward one another (e.g., pushed, compressed, or squeezed manually) such that the device 120 is in the compressed state. When in the compressed state, the first and second insertion members 320, 330 are no longer oriented parallel to one another and the top ends of the first and second insertion members 320, 330 are separated by a second distance D2 that is smaller than the first distance D1. The slot 354 can also be narrowed when the device is in the compressed state.

In some embodiments, the device 120 can be inserted into the subject (e.g., advanced towards an implantation site) while in the compressed state such that the profile of the device 120, and thus the area through which the device 120 pierces the subject's tissue, is reduced. When the device 120 (e.g., the distal end portions 321b, 331b) reaches the target site (e.g., such that a distal portion of the elongated split delivery guide 310 is positioned between vertebral bodies), the first and second pushing portions 312a, 312b can be released, allowing the elongated split delivery guide 310 to bias the first and second insertion members 320, 330 toward being separated by the first distance D1. When the device 120 returns from the compressed state to the neutral state, such as by releasing the manual squeezing of the first and second pushing portions 312a, 312b, the first and second insertion members 320, 330 can spread tissue apart or otherwise distract the spine, and the device 120 can create an enlarged space (e.g., an enlarged intervertebral space, a disc space) at the target site. Subsequently, a spinal implant or other device can be passed along the channel 350 and be delivered to the target site.

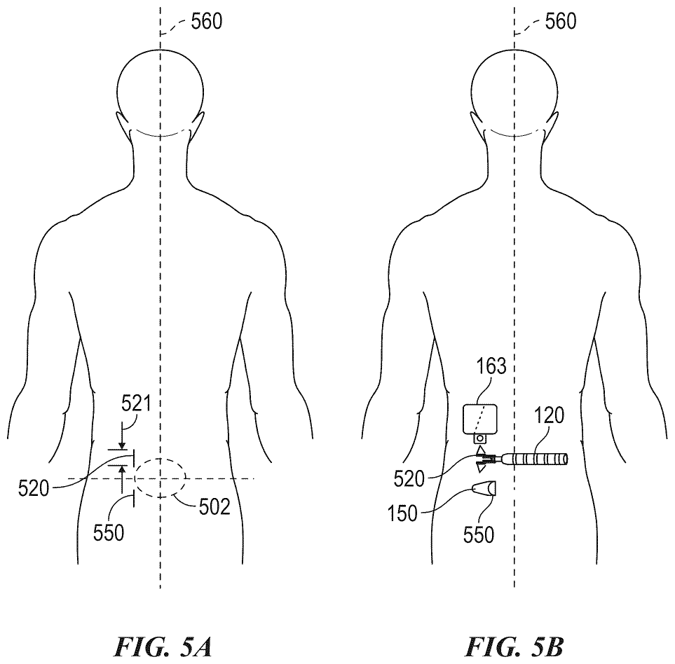

FIGS. 5A and 5B illustrate surgical steps for performing spinal procedures in accordance with embodiments of the disclosure. More specifically, FIG. 5A shows two incisions 520, 550 for accessing the left side of a lumbar spine, and a target site 502 along the subject's midsagittal plane 560. FIG. 5B shows the devices 120, 150 positioned in the incisions 520, 550, respectively. Once the devices 120, 150 are properly inserted, instruments and/or implants (not shown in FIGS. 5A and 5B) can be guided therethrough. The pillow 163 can be positioned adjacent to the devices 120, 150. For example, the illustrated pillow 163 is positioned adjacent to the device 120. The user's left hand can be supported by the pillow 163 while gripping an instrument that is guided through the devices 120, 150 for longer procedures. Additional pillows can be positioned along the patient to provide support when using the instrument.

FIGS. 6A and 6B are top views of a spinal surgical system 600 (“the system 600”) including the spinal implant delivery device 120 in different orientations in accordance with embodiments of the disclosure. Referring to both FIGS. 6A and 6B, the devices 120, 150 can be angled toward the target site 502 while maintaining a minimum distance of separation. Additional devices can be inserted into the subject to access other regions and/or provide alternative access paths. The instruments 110, 140 (illustrated in dashed line) are positioned in the devices 120, 150, respectively. In particular, the instrument 110 is shown positioned in the channel 350 of the device 150. Tissue 633 extending around the device 150 helps keep portions of the instrument 140 within the proximal ends of the device 150. The devices 120, 150 and the instruments 110, 140 can be moved (e.g., shifted, rotated) together or independently in various directions, as in indicated by arrows. The devices 120, 150 and the instruments 110, 140 can be positioned any number of times. Additionally or alternatively, additional incisions can be made along the patient to reposition the devices 120, 150 or to insert additional cannulas and instruments.

The difference between FIGS. 6A and 6B is the orientation of the device 120. In FIG. 6A, the device 120 is oriented such that the protected side, indicated by the handle assembly 340, faces the target site 502. In FIG. 6B, the device 120 is oriented such that the protected side faces away from the target site 502. On the open side of the device 120, when the instrument or implant 110 is disposed in the channel of the device 120, tissue 624 extending away from the open side of the device 120 helps keep portions of the instrument or implant 110 within the channel of the device 120. On the protected side of the device 120, the barrier formed by the second and fourth portions 324, 334 (FIG. 3E) restrict movement of the instrument or implant 110, thereby protecting adjacent tissue and/or nerves from potential damage.

The decision to orient the device 120 as shown in FIG. 6A or FIG. 6B can be based on multiple factors. For example, depending on the specific position of the target site, the type of surgical procedure involves, and/or the insertion path taken (as discussed above with reference to FIGS. 2A and 2B), a surgeon or other user may decide that either the side facing the target site 502 (as shown in FIG. 6A) or the side facing away from the target site 502 (as shown in FIG. 6B) has tissue and/or nerves that are more susceptible to damage during the procedure compared to the other side. The device 120 can be inserted in the particular orientation that protects the side determined to be more susceptible to damage.

### C. SURGICAL TECHNIQUES

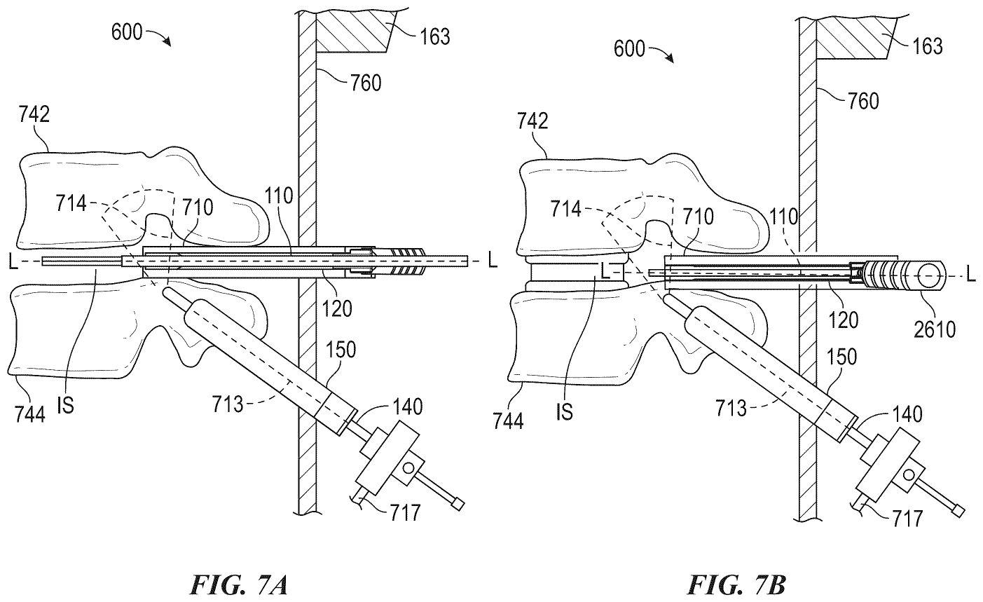

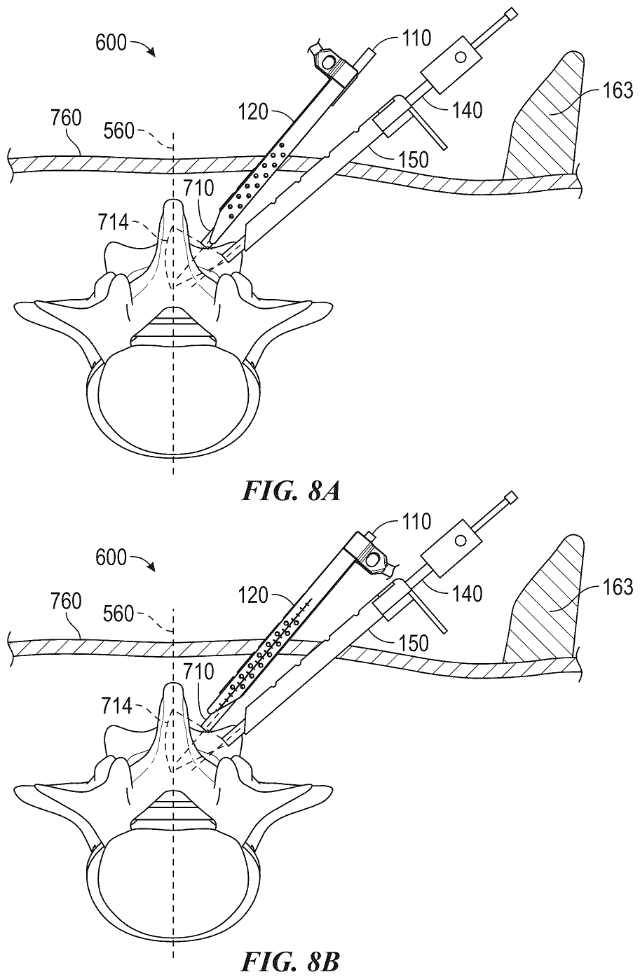

FIGS. 7A and 7B are side views of the spinal surgical system 600. FIGS. 8A and 8B are superior-to-inferior views of the spinal surgical system 600. More specifically, FIGS. 7A and 8A correspond to the orientation of the device 120 illustrated in FIG. 6A, and FIGS. 7B and 8B correspond to the orientation of the device 120 illustrated in FIG. 6B. Referring to FIGS. 7A-8B together, the devices 120, 150 extend through the subject's skin 760 (thickness not illustrated at scale) at different locations and orientations. The visualization instrument 140 has a field of view 714 suitable for viewing the spinal column and can be positioned using, for example, a transforaminal approach, a posterior approach, or a lateral approach. The pillow 163 can be positioned along the patient. An instrument holder 717 (FIGS. 7A and 7B) can hold the visualization instrument 140 at a desired position for visualization and the instrument holder 717 is spaced apart from the pillow 163. In some embodiments, the instrument holder 717 is coupled to the pillow 163.

To allow significant instrument movement, the devices 120, 150 can have axial lengths shorter than a distance from the incision in the skin 760 to the spine. The sizes of the devices 120, 150 can be selected based on the size and configuration of the incision, characteristics of the tissue, the implant to be delivered, etc. For example, the insertion members of the device 120 can be sufficiently long to extend through the subject's skin, fascia, and muscle. The channel of the device 120 can be sufficiently large to allow instruments to be inserted into and distally along the channel, which can prevent or inhibit tearing of tissue. The tissue can cover the open side of the channel to keep the instrument 110 along the device 120, and the protected side can similarly keep the instrument 110 from leaving the channel. Instruments can have relatively small diameters relative to a width of the open side of the device 120 to limit or inhibit tearing of the tissue around the incision. In some procedures, ports can be installed in some incisions and devices can be installed in other incisions without ports. A physician can determine whether to install ports based on the instruments and/or implants to be utilized and the position of the incisions. Devices, ports, and other components can be installed in each of the incisions.

The visualization instrument 140 is positioned outside intervertebral spaces to view at least a portion of an intervertebral space IS, vertebral bodies 742, 744, and/or the distal portion 710 of the instrument 110. Fluoroscopy, MR imaging, CT imaging, direct visualization, or other visualization techniques can be used in addition to or in lieu of the endoscopic viewing. Additional instruments and/or implants can be sequentially delivered through the device 120. In some procedures, multi-modality imaging of the target site can be performed using an external imaging device and the visual visualization instrument 140. The intraoperative imaging can be displayed via one or more digital screens (e.g., endoscopic imaging and fluoroscopy on different screens) in the surgical room.

FIGS. 7A-8B show the devices 120, 150 angled toward the intervertebral space IS to allow the instruments 110, 140 to be kept generally aligned with the respective devices 120, 150. The devices 120, 150 can be manually moved (e.g., shifted, rotated) between different positions and angles while inserted in the subject, as illustrated by the arrows in FIGS. 6A and 6B.

In FIGS. 7A and 8A, the open side of the device 120 faces away from the intervertebral space IS (FIG. 7A) and the subject's midsagittal plane 560 (FIG. 8A) such that the protected side of the device 120 protects the tissue and nerves between the device 120 and the spine. By contrast, in FIGS. 7B and 8B, the open side of the device 120 faces toward from the intervertebral space IS (FIG. 7B) and the subject's midsagittal plane 560 (FIG. 8B) such that the protected side of the device 120 protects the tissue and nerves away from the spine.

In some embodiments, the distal ends of the devices 120, 150 can be moved relative to the spine while the proximal ends of the devices 120, 150 remain generally stationary (e.g., at a fixed axial position relative to the skin 760). Distal portions of the instruments 110, 140 (e.g., the distal portion 710 of the instrument 110) can extend out of the distal ends of the devices 120, 150, respectively, to manipulate the tissue at the target site while shallow tissue (e.g., skin 760) retains the respective devices 120, 150.

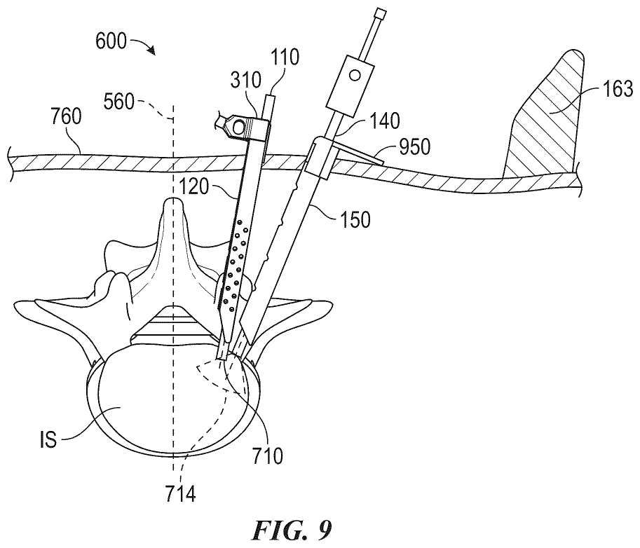

FIG. 9 is another superior-to-inferior view of the spinal surgical system 600. FIG. 9 is generally similar to FIG. 8A. For example, the devices 120, 150 and the instruments 110, 140 are inserted into the subject. The device 120 can be oriented such that the open side of the device 120 faces generally away from the subject's midsagittal plane 560 and the protected side of the device 120 protects non-targeted tissue (e.g., the tissue and nerves between the device 120 and the center of the spine). However, compared to FIG. 8A, FIG. 9 illustrates the devices 120, 150 and the instruments 110, 140 inserted deeper into the subject. In particular, each of (i) the proximal portion of the elongated split delivery guide 310 and (ii) a flange 950 of the device 150 is positioned to be adjacent to or abut against the skin 760 of the subject. Accordingly, the distal portions of the instruments 110, 140, which extend out of the distal ends of the devices 120, 150, respectively, can reach the intervertebral space IS, as shown, to manipulate the tissue thereat and/or deliver an implant thereto via, e.g., the instrument 110. For example, one or both of the distal ends of the devices 120, 150 can be positioned directly between vertebral endplates (e.g., directly between anterior, lateral, or posterior regions of the vertebral endplates), between facet joints, or the like. The positioning of the distal ends of the devices 120, 150 can be confirmed using, for example, endoscopic imaging, fluoroscopic imaging, x-rays, etc.

Referring to FIGS. 1A-9 together, the lengths of the incisions can be selected to help inhibit or limit axial rotation of the devices 120, 150. A ratio of the length of the incision to an outer width of the devices 120, 150 can be less than or equal to, for example, 1.1, 1.2, 1.3, 1.4, or 1.5. For example, a ratio of a length of the incision to a transverse width of the device 120 can be equal to or less than 1.5 to inhibit or prevent 90 degrees of rotation of the device 120 about its longitudinal axis L-L. This keeps the desired orientation of the device 120 during a procedure. Instrument holders can hold the devices 120, 150, instruments 110, 140, or a combination of devices and instruments. The instrument holders can be particularly useful when performing surgical techniques involving three or more ports.

Instruments can be selected to treat, without limitation, spinal nerve compression (e.g., spinal cord compression, spinal nerve root compression, or the like), spinal disc herniation, osteoporosis, stenosis, or other diseases or conditions. After accessing the work space, the tissue removal tip can remove unwanted tissue, including, without limitation, tissue bulging from discs, bone (e.g., lamina, lateral recesses, facets including the inferior facets, etc.), bone spurs (e.g., bone spurs associated with osteoarthritis), tissue of thickened ligaments, spinal tumors, displaced tissue (e.g., tissue displaced by a spinal injury), or tissue that may cause or contribute to spinal nerve compression. The instrument, as well as other instruments (e.g., rongeurs, debulkers, scrapers, reamers, dilators, etc.), can be used to perform one or more dilation procedures, decompression procedures, discectomies, microdiscectomies, laminotomies, or combinations thereof. In procedures for treating stenosis, the instrument can be used to remove tissue associated with central canal stenosis, lateral recess stenosis, and/or other types of stenosis. In some decompression procedures, the instrument can be a tissue removal device used to, for example, remove bone, separate the ligamentum flavum from one or both vertebrae, cut or debulk the ligamentum flavum, remove loose tissue, and remove at least a portion of the intervertebral disc. Each stage can be performed with a different instrument.

As discussed above, the visualization instrument 140 can be, without limitation, an endoscopic instrument that includes fiber optics suitable to image the treatment site and surrounding tissues, such as the spinal cord, nerves branching from spinal cord, ligaments, vertebrae 742, 744, the intervertebral space IS, or any other features or anatomical structures of interest while an implant is delivered through the device 120. Surrounding non-targeted tissue can be viewed to ensure that the distal tip 710 does not injure it. This allows a physician to remove tissue and/or deliver implants without damaging nerve tissue, the spinal cord, and other non-targeted tissue.

In some embodiments, the visualization instrument 140 can be a steerable to facilitate navigation around anatomical features. The visualization instrument 140 can include a fiber-optic scope or a flexible or rigid instrument with one or more illumination elements (e.g., fiber optics for illumination) or imaging elements (e.g., charge-coupled devices for imaging) suitable for visualizing the interior of otherwise inaccessible sites. In some embodiments, the visualization instrument 140 can be rod-lens endoscopes with an outer diameter equal to or smaller than about 2 mm, 3 mm, 4 mm, 5 mm, 6 mm, 8 mm, or 10 mm, and a length equal to or shorter than about 15 cm, 20 cm, 30 cm, or 40 cm. The visualization instruments 140 can also have integrated irrigation features (e.g., valves, flow control buttons, fluid lumens, return lumens), connectors (e.g., electrical connectors, fluidic connectors, etc.), access ports (e.g., access ports connected to lumens, such as lumens through which instruments can pass), or the like. In embodiments with an angled lens, the visualization instrument can have approximately 0-degree, 10-degree, 15-degree, 30-degree, or 45-degree lens angles, which are toward a light source. In other angled lens embodiments, the visualization instrument can have an approximately 15-degree, 30-degree, or 45-degree lens angled away from a light source. The angle of the lens can be selected based on the area to be viewed. In some posterior or lateral spinal procedures, a 0-degree lens can provide a wide-angle view suitable for viewing nerve roots, the spinal cord, and intervertebral space. A 30- or 45-degree lens endoscope angled toward the light source can be used to provide an angled view toward, for example, the spine or midsagittal plane to view, for example, the spinous processes, spinal cord, or central regions of the intervertebral space. A 30- or 45-degree lens endoscope angled away from the light source can be used to provide an angled view toward the lateral features or the spine, such as nerve roots at the neural foramen, side regions of the intervertebral space, or the like.

### D. EXAMPLES

The present technology is illustrated, for example, according to various aspects described below as numbered examples (1, 2, 3, etc.) for convenience. These are provided as examples and do not limit the present technology. It is noted that any of the dependent examples may be combined in any combination, and placed into a respective independent example. The other examples can be presented in a similar manner.

1. A method for delivering a spinal implant to a subject's spine, the method comprising:
inserting an implant delivery device in a subject, wherein the implant delivery device includes:
   a handle assembly; and
   an elongated split delivery guide coupled to the handle assembly and defining a delivery channel, wherein the elongated split delivery guide includes first and second insertion members defining a longitudinally-extending slot positioned along a bottom of the delivery channel, wherein the first and second insertion members are movable toward each other to narrow the delivery channel for advancing the elongated split delivery guide toward an implantation site in a patient; and
delivering a spinal implant to the subject's spine along the delivery channel toward the implantation site while the first and second insertion members keep the implant within the channel.

2. The method of example 1, wherein:
the first insertion member extends from the handle assembly and includes a first bottom and a first sidewall extending substantially perpendicular to the first bottom,
the second insertion member extends from the handle assembly and spaced apart from the first insertion member,
the second insertion member includes a second bottom and a second sidewall extending substantially perpendicular to the second bottom and substantially parallel to the first sidewall, and
the first and second bottoms extend toward each other and define the slot.

3. The method of example 1 or example 2, wherein, the elongated split delivery guide is biased to keep the first and second insertion members spaced apart by a first distance, and wherein the method further comprises:
compressing, prior to inserting the device in the subject, the device such that the first and second insertion members are spaced apart by a second distance smaller than the first distance;
releasing, after inserting the device in the subject, the device such that the elongated split delivery guide biases the first and second insertion members toward being spaced apart by the first distance.

4. The method of any one of examples 1-3, wherein:
the first insertion member includes distance indicators and a plurality of apertures arranged along the distance indicators,
inserting the device in the subject comprises inserting device by a predetermined distance based on the distance indicators, and
delivering the spinal implant comprises determining a position of the spinal implant based on a view of the spinal implant through the plurality of apertures.

5. The method of any one of examples 1-4, further comprising moving the spinal implant along the elongated split delivery guide while a distal portion of the elongated split delivery guide is positioned directly between vertebral bodies of the subject's spine.

6. The method of any one of examples 1-5, further comprising:
positioning a distal portion, in a compressed configuration, of the elongated split delivery guide between vertebral bodies of the subject's spine; and
allowing expansion of the distal portion toward an expanded configuration to distract the subject's spine.

7. A method for accessing an intervertebral space of a subject's spine, the method comprising:
inserting a delivery device into a subject, the delivery device including:
   a handle assembly, and
   an elongated split delivery guide coupled to the handle assembly, wherein the elongated split delivery guide includes a first insertion member and a second insertion member each having a sidewall portion and a bottom portion extending from the sidewall portion in a substantially perpendicular direction, wherein each of the first insertion member and the second insertion member is cantilevered and coupled to one another at a proximal portion of the elongated split delivery guide, wherein the sidewall portion of the first insertion member and the sidewall portion of the second insertion member are biased to extend parallel to one another, and wherein the sidewall portions and the bottom portions of the first and second insertion members define a delivery channel of the elongated split delivery guide; and
passing a surgical device along the delivery channel of the elongated split delivery guide and toward an intervertebral space of the subject.

8. The method of example 7, wherein the elongated split delivery guide includes (i) a protected face defined by the bottom portions of the first and second insertion members and (ii) an open side opposite the protected face, and wherein inserting the delivery device comprises orienting the delivery device such that the protected face faces the intervertebral space of the subject when the delivery device is inserted in the subject.

9. The method of example 7, wherein the elongated split delivery guide includes (i) a protected face defined by the bottom portions of the first and second insertion members and (ii) an open side opposite the protected face, and wherein inserting the delivery device comprises orienting the delivery device such that the open side faces the intervertebral space of the subject when the delivery device is inserted in the subject.

10. The method of any one of examples 7-9, further comprising:
compressing, prior to inserting the delivery device in the subject, the proximal portion of the elongated split delivery guide such that the first and second insertion members move toward one another, thereby reducing a profile of the elongated split delivery guide,
wherein inserting comprises inserting the delivery device into the subject while the delivery device is compressed.

11. The method of example 10, wherein inserting comprises inserting the delivery device between adjacent vertebral endplates of the subject, and wherein the method further comprises:
releasing, after inserting the delivery device in the subject, the proximal portion of the elongated split delivery guide such that the proximal portion becomes uncompressed and the first and second insertion members move away from one another, thereby spreading the adjacent vertebral endplates apart and expanding the profile of the elongated split delivery guide.

12. The method of any one of examples 7-11, further comprising:
inserting a visualization instrument in the subject such that a distal tip of the visualization instrument is positioned adjacent to the first insertion member of the elongated split delivery guide; and
visualizing, via the visualization instrument and a plurality of apertures arranged along a length of the first insertion member, the surgical device passing along the delivery channel of the elongated split delivery guide.

13. The method of example 12, further comprising:
determining, via the visualization instrument and a plurality of depth markings arranged along the length of the first insertion member, a position of the surgical device relative to the subject.

14. The method of any one of examples 7-13, wherein inserting the delivery device comprises inserting the delivery device until a distal tip of the elongated split delivery guide is positioned in the intervertebral space of the subject.

15. The method of any one of examples 7-14, wherein inserting the delivery device comprises inserting the delivery device until the proximal portion of the elongated split delivery guide abuts against a skin of the subject.

16. The method of any one of examples 7-15, wherein passing the surgical device comprises passing a spinal implant along the delivery channel of the elongated split delivery guide and to the intervertebral space of the subject.

17. A spinal implant delivery device, the device comprising:
a handle assembly;
a first insertion member extending from the handle assembly in an insertion direction, wherein the first insertion member includes a first portion and a second portion extending substantially perpendicular to the first portion; and
a second insertion member extending from the handle assembly in the insertion direction and spaced apart from the first insertion, wherein the second insertion member includes a third portion and a fourth portion extending substantially perpendicular to the third portion,
wherein the second and fourth portions extend toward each other, and
wherein the first, second, third, and fourth portions at least partially define a channel through which a spinal implant can be delivered to a subject's spine.

18. The device of example 17, wherein the first portion includes distance indicators along the direction and a plurality of apertures arranged along the distance indicators.

19. The device of example 17 or example 18, wherein the first portion is made from a transparent or translucent material, and wherein the first portion includes distance indicators along the insertion direction.

20. The device of any one of examples 17-19, wherein the first and third portions are biased to extend parallel to one another, and wherein the first and second insertion members are configured to be pushed toward one another such that the first and third portions are no longer parallel to one another, thereby reducing a profile of the device.

21. The device of any one of examples 17-20, wherein the second and fourth portions define a protected side extending along lengths of the first and second insertion members, and wherein the handle assembly is oriented on the protected side.

22. The device of any one of examples 17-21, wherein the second and fourth portions are spaced apart to define a gap therebetween.

23. The device of any one of examples 17-22, wherein each of the first and second insertion members is cantilevered.

24. A spinal implant delivery system, comprising:
a handle assembly; and
an implant delivery guide coupled to the handle assembly and including:
   a slotted channel member having a compressed configuration for insertion into a disc space between vertebral bodies of a patient and an expanded configuration for distracting the vertebral bodies, and
   an implant delivery channel defined by the slotted channel member and configured to allow an implant to be delivered into the disc space when the slotted channel member is in the expanded configuration.

25. The spinal implant delivery system of example 24, wherein the implant delivery guide has a pair of cantilevered fingers each having a generally L-shaped cross section.

26. The spinal implant delivery system of example 24 or example 25, wherein the implant delivery guide has a longitudinal axis and a U-shaped cross-section taken perpendicular to the longitudinal axis.

27. The spinal implant delivery system of any one of examples 24-26, further comprising an expandable intervertebral implant having a collapsed state for moving along the implant delivery channel and an expanded state for contacting the vertebral bodies, wherein the expandable intervertebral implant expands outwardly past sidewalls of the implant delivery guide when moved from the collapsed state to the expanded state.

28. The spinal implant delivery system of any one of examples 24-27, wherein the implant delivery guide includes first and second cantilevered insertion members each having tapered distal ends configured to lay flat against the vertebral bodies while a slotted region of the slotted channel member faces a midsagittal plane of the patient.

29. The spinal implant delivery system of any one of examples 24-28, wherein the implant delivery guide includes:
a first insertion member extending from the handle assembly in an insertion direction, wherein the first insertion member includes a first portion and a second portion extending substantially perpendicular to the first portion; and
a second insertion member extending from the handle assembly in the insertion direction and spaced apart from the first insertion, wherein the second insertion member includes a third portion and a fourth portion extending substantially perpendicular to the third portion,
wherein the second and fourth portions extend toward each other, and wherein the first, second, third, and fourth portions at least partially define the implant delivery channel.

30. The spinal implant delivery system of any one of examples 24-29, wherein the slotted channel member includes distance indicators spaced apart along a longitudinal axis of the slotted channel member and a plurality of apertures arranged along the distance indicators.

31. The spinal implant delivery system of any one of examples 24-30, wherein the slotted channel member comprises a transparent or translucent material.

32. The spinal implant delivery system of any one of examples 24-31, wherein the slotted channel member has first and second sidewalls extending parallel to one another when the slotted channel is in the expanded configuration and extending at a non-parallel orientation relative to one another when the slotted channel is in the compressed configuration.

33. The spinal implant delivery system of any one of examples 24-32, wherein the slotted channel member is configured to move spaced apart distal tapered ends of the slotted channel member towards one another when a proximal end of the slotted channel member is compressed.

34. The spinal implant delivery system of any one of examples 24-33, wherein the slotted channel member includes a pair of tapered distal sidewalls spaced apart from one another.

35. The spinal implant delivery system of any one of examples 24-34, wherein the slotted channel member includes first and second insertion members are configured to be pushed toward one another to reduce a width of a slot between the first and second insertion members.

36. The spinal implant delivery system of any one of examples 24-35, wherein the implant delivery guide includes a pair of members at a bottom of the implant delivery channel, wherein the pair of members defines a slot.

### E. CONCLUSION

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one skilled in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal-bearing medium used to actually carry out the distribution. Examples of a signal-bearing medium include, but are not limited to, the following: a recordable type of medium such as a floppy disk, a hard disk drive, a CD, a DVD, a digital tape, a computer memory, etc.; and a transmission type medium such as a digital and/or an analog communication medium (e.g., a fiber-optic cable, a waveguide, a wired communications link, a wireless communication link, etc.).

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Features from various systems, methods, and instruments can be combined with features disclosed in U.S. App. No. 63/611,913; U.S. application Ser. No. 15/793,950; U.S. application Ser. No. 17/902,685; U.S. application Ser. No. 18/335,737; U.S. application Ser. No. 18/464,949; U.S. application Ser. No. 18/470,140; U.S. application Ser. No. 18/764,784; U.S. Pat. Nos. 8,632,594; 9,308,099; 9,820,788; 10,105,238; 10,201,431; 10,322,009; 10,898,340; 11,464,648; 11,950,770; PCT App. No. PCT/US20/49982; and PCT App. No. PCT/US22/21193, which are hereby incorporated by reference and made a part of this application. Variations of the implants are contemplated.

Systems, components, and instruments disclosed herein can be disposable or reusable. For example, the ports, instruments, or cannulas can be disposable to prevent cross-contamination. As used herein, the term “disposable” when applied to a system or component (or combination of components), such as an instrument, a tool, or a distal tip or a head, is a broad term and generally means, without limitation, that the system or component in question is used a finite number of times and is then discarded. Some disposable components are used only once and are then discarded. In other embodiments, the components and instruments are non-disposable and can be used any number of times. In some kits, all of the components can be disposable to prevent cross-contamination. In some other kits, components (e.g., all or some of the components) can be reusable.

The term “substantially” as used herein means within 10% of the stated value. For example, a first component that extends “substantially perpendicular” from a second component can be oriented at an angle between 81-99 degrees relative to the second component. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

What is claimed is:

1. A method for delivering a spinal implant to a subject's spine, the method comprising:
inserting an implant delivery device in a subject, wherein the implant delivery device includes:
a handle assembly; and
an elongated split delivery guide coupled to the handle assembly and defining a delivery channel, wherein the elongated split delivery guide includes first and second insertion members defining a longitudinally-extending slot positioned along a bottom of the delivery channel, wherein the first and second insertion members are movable toward each other to narrow the delivery channel for advancing the elongated split delivery guide toward an implantation site in a patient; and
delivering a spinal implant to the subject's spine along the delivery channel toward the implantation site while the first and second insertion members keep the spinal implant within the channel.

2. The method of claim 1, wherein:
the first insertion member extends from the handle assembly and includes a first bottom and a first sidewall extending substantially perpendicular to the first bottom,
the second insertion member extends from the handle assembly and spaced apart from the first insertion member,
the second insertion member includes a second bottom and a second sidewall extending substantially perpendicular to the second bottom and substantially parallel to the first sidewall, and
the first and second bottoms extend toward each other and define the slot.

3. The method of claim 1, wherein, the elongated split delivery guide is biased to keep the first and second insertion members spaced apart by a first distance, and wherein the method further comprises:
compressing, prior to inserting the device in the subject, the device such that the first and second insertion members are spaced apart by a second distance smaller than the first distance;
releasing, after inserting the device in the subject, the device such that the elongated split delivery guide biases the first and second insertion members toward being spaced apart by the first distance.

4. The method of claim 1, wherein:
the first insertion member includes distance indicators and a plurality of apertures arranged along the distance indicators,
inserting the device in the subject comprises inserting device by a predetermined distance based on the distance indicators, and
delivering the spinal implant comprises determining a position of the spinal implant based on a view of the spinal implant through the plurality of apertures.

5. The method of claim 1, further comprising moving the spinal implant along the elongated split delivery guide while a distal portion of the elongated split delivery guide is positioned directly between vertebral bodies of the subject's spine.

6. The method of claim 1, further comprising:
positioning a distal portion, in a compressed configuration, of the elongated split delivery guide between vertebral bodies of the subject's spine; and
allowing expansion of the distal portion toward an expanded configuration to distract the subject's spine.

7. A method for accessing an intervertebral space of a subject's spine, the method comprising:
inserting a delivery device into a subject, the delivery device including:
a handle assembly, and
an elongated split delivery guide coupled to the handle assembly, wherein the elongated split delivery guide includes a first insertion member and a second insertion member each having a sidewall portion and a bottom portion extending from the sidewall portion in a substantially perpendicular direction, wherein each of the first insertion member and the second insertion member is cantilevered and coupled to one another at a proximal portion of the elongated split delivery guide, wherein the sidewall portion of the first insertion member and the sidewall portion of the second insertion member are biased to extend parallel to one another, and wherein the sidewall portions and the bottom portions of the first and second insertion members define a delivery channel of the elongated split delivery guide; and
passing a surgical device along the delivery channel of the elongated split delivery guide and toward an intervertebral space of the subject.

8. The method of claim 7, wherein the elongated split delivery guide includes (i) a protected face defined by the bottom portions of the first and second insertion members and (ii) an open side opposite the protected face, and wherein inserting the delivery device comprises orienting the delivery device such that the protected face faces the intervertebral space of the subject when the delivery device is inserted in the subject.

9. The method of claim 7, wherein the elongated split delivery guide includes (i) a protected face defined by the bottom portions of the first and second insertion members and (ii) an open side opposite the protected face, and wherein inserting the delivery device comprises orienting the delivery device such that the open side faces the intervertebral space of the subject when the delivery device is inserted in the subject.

10. The method of claim 7, further comprising:
compressing, prior to inserting the delivery device in the subject, the proximal portion of the elongated split delivery guide such that the first and second insertion members move toward one another, thereby reducing a profile of the elongated split delivery guide,
wherein inserting comprises inserting the delivery device into the subject while the delivery device is compressed.

11. The method of claim 10, wherein inserting comprises inserting the delivery device between adjacent vertebral endplates of the subject, and wherein the method further comprises:
releasing, after inserting the delivery device in the subject, the proximal portion of the elongated split delivery guide such that the proximal portion becomes uncompressed and the first and second insertion members move away from one another, thereby spreading the adjacent vertebral endplates apart and expanding the profile of the elongated split delivery guide.

12. The method of claim 7, further comprising:
inserting a visualization instrument in the subject such that a distal tip of the visualization instrument is positioned adjacent to the first insertion member of the elongated split delivery guide; and
visualizing, via the visualization instrument and a plurality of apertures arranged along a length of the first insertion member, the surgical device passing along the delivery channel of the elongated split delivery guide.

13. The method of claim 12, further comprising:
determining, via the visualization instrument and a plurality of depth markings arranged along the length of the first insertion member, a position of the surgical device relative to the subject.

14. The method of claim 7, wherein inserting the delivery device comprises inserting the delivery device until a distal tip of the elongated split delivery guide is positioned in the intervertebral space of the subject.

15. The method of claim 7, wherein inserting the delivery device comprises inserting the delivery device until the proximal portion of the elongated split delivery guide abuts against a skin of the subject.

16. The method of claim 7, wherein passing the surgical device comprises passing a spinal implant along the delivery channel of the elongated split delivery guide and to the intervertebral space of the subject.

17. A spinal implant delivery device, the device comprising:
a handle assembly;
a first insertion member extending from the handle assembly in an insertion direction, wherein the first insertion member includes a first portion and a second portion extending substantially perpendicular to the first portion; and
a second insertion member extending from the handle assembly in the insertion direction and spaced apart from the first insertion, wherein the second insertion member includes a third portion and a fourth portion extending substantially perpendicular to the third portion,
wherein the second and fourth portions extend toward each other, and
wherein the first, second, third, and fourth portions at least partially define a channel through which a spinal implant can be delivered to a subject's spine.

18. The spinal implant delivery device of claim 17, wherein the first portion includes distance indicators along the direction and a plurality of apertures arranged along the distance indicators.

19. The spinal implant delivery device of claim 17, wherein the first portion is made from a transparent or translucent material, and wherein the first portion includes distance indicators along the insertion direction.

20. The spinal implant delivery device of claim 17, wherein the first and third portions are biased to extend parallel to one another, and wherein the first and second insertion members are configured to be pushed toward one another such that the first and third portions are no longer parallel to one another, thereby reducing a profile of the device.

21. The spinal implant delivery device of claim 17, wherein the second and fourth portions define a protected side extending along lengths of the first and second insertion members, and wherein the handle assembly is oriented on the protected side.

22. The spinal implant delivery device of claim 17, wherein the second and fourth portions are spaced apart to define a gap therebetween.

23. The spinal implant delivery device of claim 17, wherein each of the first and second insertion members is cantilevered.

24. A spinal implant delivery system, comprising:
a handle assembly; and
an implant delivery guide coupled to the handle assembly and including:
a slotted channel member having a compressed configuration for insertion into a disc space between vertebral bodies of a patient and an expanded configuration for distracting the vertebral bodies, and
an implant delivery channel defined by the slotted channel member and configured to allow an implant to be delivered into the disc space when the slotted channel member is in the expanded configuration.

25. The spinal implant delivery system of claim 24, wherein the implant delivery guide has a pair of cantilevered fingers each having a generally L-shaped cross section.

26. The spinal implant delivery system of claim 24, wherein the implant delivery guide has a longitudinal axis and a U-shaped cross-section taken perpendicular to the longitudinal axis.

27. The spinal implant delivery system of claim 24, further comprising an expandable intervertebral implant having a collapsed state for moving along the implant delivery channel and an expanded state for contacting the vertebral bodies, wherein the expandable intervertebral implant expands outwardly past sidewalls of the implant delivery guide when moved from the collapsed state to the expanded state.

28. The spinal implant delivery system of claim 24, wherein the implant delivery guide includes first and second cantilevered insertion members each having tapered distal ends configured to lay flat against the vertebral bodies while a slotted region of the slotted channel member faces a midsagittal plane of the patient.

29. The spinal implant delivery system of claim 24, wherein the implant delivery guide includes:
a first insertion member extending from the handle assembly in an insertion direction, wherein the first insertion member includes a first portion and a second portion extending substantially perpendicular to the first portion; and
a second insertion member extending from the handle assembly in the insertion direction and spaced apart from the first insertion, wherein the second insertion member includes a third portion and a fourth portion extending substantially perpendicular to the third portion,
wherein the second and fourth portions extend toward each other, and wherein the first, second, third, and fourth portions at least partially define the implant delivery channel.

30. The spinal implant delivery system of claim 24, wherein the slotted channel member includes distance indicators spaced apart along a longitudinal axis of the slotted channel member and a plurality of apertures arranged along the distance indicators.

31. The spinal implant delivery system of claim 24, wherein the slotted channel member comprises a transparent or translucent material.

32. The spinal implant delivery system of claim 24, wherein the slotted channel member has first and second sidewalls extending parallel to one another when the slotted channel is in the expanded configuration and extending at a non-parallel orientation relative to one another when the slotted channel is in the compressed configuration.

33. The spinal implant delivery system of claim 24, wherein the slotted channel member is configured to move spaced apart distal tapered ends of the slotted channel member towards one another when a proximal end of the slotted channel member is compressed.

34. The spinal implant delivery system of claim 24, wherein the slotted channel member includes a pair of tapered distal sidewalls spaced apart from one another.

35. The spinal implant delivery system of claim 24, wherein the slotted channel member includes first and second insertion members are configured to be pushed toward one another to reduce a width of a slot between the first and second insertion members.

36. The spinal implant delivery system of claim 24, wherein the implant delivery guide includes a pair of members at a bottom of the implant delivery channel, wherein the pair of members defines a slot.
